SEQUENCE LISTING (32 sequences)

SEQ ID NO: 1 - DNA, 807 bp, Paraburkholderia xenovorans
  source 1..807: mol_type = other DNA
atgtatctgttgagctatccggaacaggttgattatccgatgtcctatgccatttatccgagcttgtccggtaaaaccgttgttattaccggtggtggttccggtattggtgccgctatggttgaagcctttgcccgtcagggtgctcgtgttttcttccttgatgttgctgaagatgattctttggctttgcagcagtctttgtctgatgctccgcatccgccgttgtttcgtcgttgtgatcttcgttctgttgatgctattcattctgcttttgccggtattgttgaaattgccggtcctattgaagttttggttaataatgccggtaatgatgatcgtcatgaagttgatgctatcacccctgcctattgggatgaacgtatggctgttaatttgcgtcatcagttcttctgtgcccaggcggctgcggctggtatgcgtaaaatcggtcgtggtgttattttgaatttgggctctgtttcttggcatttggctttgccgaatttggctatttatatgtctgccaaagctggtattgaaggtttgacgcgtggtcttgcccgtgatttgggtgctgccggtatccgtgttaattgtattattccgggtgccgttcgtacgccgcgtcaaatgcagttgtggcagtctccggaatctgaagccaaattggttgcctctcagtgtttgcgtttgcgtattgaacctgaacatgttgcccgtatggctttgtttttggcctctgatgatgcgtctcgttgttctggtcgtgattattttgttgatgctggttggtatggtgaataa

SEQ ID NO: 2 - DNA, 199 bp, unidentified
  source 1..199: mol_type = other DNA
tatcgctcatgatcgcggcatgttctgatatttttcctctaaaaaagataaaaagtcttttcgcttcggcagaagaggttcatcatgaacaaaaattcggcatttttaaaaatgcctatagctaaatccggaacgacactttagaggtttctgggtcatcctgattcagacatagtgttttgaatatatggagtaagca

SEQ ID NO: 3 - DNA, 41 bp, synthetic construct
  source 1..41: mol_type = other DNA
attcgcggccgcttctagagtatcgctcatgatcgcggcat

SEQ ID NO: 4 - DNA, 57 bp, unidentified
  source 1..57: mol_type = other DNA
ggatagctcaacagatacattgcttactccatatattcaaaacactatgtctgaatc

SEQ ID NO: 5 - DNA, 27 bp, synthetic construct
  source 1..27: mol_type = other DNA
atgtatctgttgagctatccggaacag

SEQ ID NO: 6 - DNA, 31 bp, synthetic construct
  source 1..31: mol_type = other DNA
ttattcaccataccaaccagcatcaacaaaa

SEQ ID NO: 7 - DNA, 17 bp, synthetic construct
  source 1..17: mol_type = other DNA
gtaaaacgacggccagt

SEQ ID NO: 8 - DNA, 17 bp, synthetic construct
  source 1..17: mol_type = other DNA
gtcatagctgtttcctg

SEQ ID NO: 9 - DNA, 1968 bp, Escherichia coli
  source 1..1968: mol_type = other DNA
atgaccattgagaaaattttcaccccgcaggacgacgcgttttatgcggtgatcacccacgcggcggggccgcagggcgctctgccgctgaccccgcagatgctgatggaatctcccagcggcaacctgttcggcatgacgcagaacgccgggatgggctgggacgccaacaagctcaccggcaaagaggtgctgattatcggcactcagggcggcatccgcgccggagacggacgcccaatcgcgctgggctaccacaccgggcattgggagatcggcatgcagatgcaggcggcggcgaaggagatcacccgcaatggcgggatcccgttcgcggccttcgtcagcgatccgtgcgacgggcgctcgcagggcacgcacggtatgttcgattccctgccgtaccgcaacgacgcggcgatcgtgtttcgccgcctgatccgctccctgccgacgcggcgggcggtgatcggcgtagcgacctgcgataaagggctgcccgccaccatgattgcgctggccgcgatgcacgacctgccgactattctggtgccgggcggggcgacgctgccgccgaccgtcggggaagacgcgggcaaggtgcagaccatcggcgcgcgtttcgccaaccacgaactctccctgcaggaggccgccgaactgggctgtcgcgcctgcgcctcgccgggcggcgggtgtcagttcctcggcacggcgggcacctcgcaggtggtcgcggaggcgctgggtctggcgctgccgcactccgcgctggcgccgtccgggcaggcggtgtggctggagatcgcccgccagtcggcgcgcgcggtcagcgagctggatagccgcggcatcaccacgcgggatatcctctccgataaagccatcgaaaacgcgatggtgatccacgcggcgttcggcggctccaccaatttactgctgcacattccggccatcgcccacgcggcgggctgcacgatcccggacgttgagcactggacgcgcatcaaccgtaaagtgccgcgtctggtgagcgtgctgcccaacggcccggactatcacccgaccgtgcgcgccttcctcgcgggcggcgtgccggaggtgatgctccacctgcgcgacctcggcctgctgcatctggacgccatgaccgtgaccggccagacggtgggcgagaaccttgaatggtggcaggcgtccgagcgccgggcgcgcttccgccagtgcctgcgcgagcaggacggcgtagagccggatgacgtgatcctgccgccggagaaggcaaaagcgaaagggctgacctcgacggtctgcttcccgacgggcaacatcgctccggaaggttcggtgatcaaggccacggcgatcgacccgtcggtggtgggcgaagatggcgtataccaccacaccggccgggtgcgggtgtttgtctcggaagcgcaggcgatcaaggcgatcaagcgggaagagattgtgcagggcgatatcatggtggtgatcggcggcgggccgtccggcaccggcatggaagagacctaccagctcacctccgcgctaaagcatatctcgtggggcaagacggtgtcgctcatcaccgatgcgcgcttctcgggcgtgtcgacgggcgcctgcttcggccacgtgtcgccggaggcgctggcgggcgggccgattggcaagctgcgcgataacgacatcatcgagattgccgtggatcgtctgacgttaactggcagcgtgaacttcatcggcaccgcggacaacccgctgacgccggaagagggcgcgcgcgagctggcgcggcggcagacgcacccggacctgcacgcccacgactttttgccggacgacacccggctgtgggcggcactgcagtcggtgagcggcggcacctggaaaggctgtatttatgacaccgataaaattatcgaggtaattaacgccggtaaaaaagcgctcggaatttaa

SEQ ID NO: 10 - DNA, 909 bp, unidentified
  source 1..909: mol_type = other DNA
atgccgcagtccgcgttgttcacgggaatcattccccctgtctccaccatttttaccgccgacggccagctcgataagccgggcaccgccgcgctgatcgacgatctgatcaaagcaggcgttgacggcctgttcttcctgggcagcggtggcgagttctcccagctcggcgccgaagagcgtaaagccattgcccgctttgctatcgatcatgtcgatcgtcgcgtgccggtgctgatcggcaccggcggcaccaacgcccgggaaaccatcgaactcagccagcacgcgcagcaggcgggcgcggacggcatcgtggtgatcaacccctactactggaaagtgtcggaagcgaacctgatccgctatttcgagcaggtggccgacagcgtcacgctgccggtgatgctctataacttcccggcgctgaccgggcaggatctgactccggcgctggtgaaaaccctcgccgactcgcgcagcaatattatcggcatcaaagacaccatcgactccgtcgcccacctgcgcagcatgatccataccgtcaaaggtgcccatccgcacttcaccgtgctctgcggctacgacgatcatctgttcaataccctgctgctcggcggcgacggggcgatatcggcgagcggcaactttgccccgcaggtgtcggtgaatcttctgaaagcctggcgcgacggggacgtggcgaaagcggccgggtatcatcagaccttgctgcaaattccgcagatgtatcagctggatacgccgtttgtgaacgtgattaaagaggcgatcgtgctctgcggtcgtcctgtctccacgcacgtgctgccgcccgcctcgccgctggacgagccgcgcaaggcgcagctgaaaaccctgctgcaacagctcaagctttgctga

SEQ ID NO: 11 - DNA, 1164 bp, Escherichia coli
  source 1..1164: mol_type = other DNA
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 12 - DNA, 1776 bp, unidentified
  source 1..1776: mol_type = other DNA
atgagcaatcgcaccccgcgtcgttttcgcagccgtgattggtttgataatccggatcatattgatatgaccgccttgtatttggaacgctttatgaattatggtatcaccccggaagaattacgttctggcaaacctattattggtattgcccagacgggttctgatatttctccttgtaatcgtattcatttggatttggttcagcgggttcgggatggtattcgcgatgccggtggtattcctatggaatttccggttcatcctattttcgaaaattgtcgtcgtccgacggccgccttagatcgtaatttgtcttatttgggtttggttgaaacgttgcatggttatccgattgatgccgttgttttgaccaccggttgtgataaaaccacccctgccggtattatggctgccaccaccgttaatattcctgccattgttctttctggtggcccgatgttggatggttggcatgaaaatgaattggttggctctggcacggtcatttggcgttctcgtcgtaaattggccgccggtgaaatcaccgaagaagaatttattgatcgtgctgcttcttctgctccgtctgccggtcattgtaataccatgggtaccgcctctaccatgaatgccgttgccgaagccttgggtttgtctttaaccggttgtgctgccattccggccccttatcgtgaacgtggtcagatggcctataaaaccggccagcgtattgtcgatttggcgtatgatgatgttaaaccgttggatattttgaccaaacaggcttttgaaaatgctattgccttggttgccgccgctggtggttctaccaatgcccaaccgcatattgttgccatggcccgtcatgctggtgtcgaaattaccgccgatgattggcgtgccgcttatgatattccgttgattgttaatatgcagccggcgggtaaatatttaggtgaacgttttcatcgtgcgggtggtgcccctgctgttttgtgggaattgttgcagcagggtcgtttgcatggtgatgttttgacggttacgggtaaaaccatgtctgaaaatttgcagggtcgtgaaacctctgatcgtgaagttattttcccgtatcatgaaccgcttgccgaaaaggccggttttttggttttgaaaggtaatttgtttgattttgccatcatgaaaagctctgttattggtgaagaatttcgtaaacgttatttgtctcagccgggtcaggaaggtgttttcgaagctcgtgccattgttttcgatggttctgatgattatcataaacgtatcaatgatccggccttggaaattgatgaacgctgtattttggtcattcgtggtgccggtccgattggttggccgggttctgctgaagttgttaatatgcaaccgccggatcatttgttgaagaagggtattatgtctttgccgaccttgggtgatggtcgtcagtctggtacggccgattctccgtctattttgaatgcctctcctgaaagcgccatcggtggtggtttatcttggttgcgtacgggtgataccattcgtattgatttgaatacgggtcgttgtgatgccttggttgatgaagccaccattgccgcccgcaaacaggatggtattccggctgttcctgctacgatgaccccgtggcaagaaatttatcgtgctcatgccagccagttagatacgggtggtgttttggaatttgccgtcaaatatcaagatttggcggctaaattgccgcgtcataatcattaa

SEQ ID NO: 13 - DNA, 1968 bp, Escherichia coli
  source 1..1968: mol_type = other DNA
atgtctgttcgcaatatttttgctgacgagagccacgatatttacaccgtcagaacgcacgccgatggcccggacggcgaactcccattaaccgcagagatgcttatcaaccgcccgagcggggatctgttcggtatgaccatgaatgccggaatgggttggtctccggacgagctggatcgggacggtattttactgctcagtacactcggtggcttacgcggcgcagacggtaaacccgtggcgctggcgttgcaccaggggcattacgaactggacatccagatgaaagcggcggccgaggttattaaagccaaccatgccctgccctatgccgtgtacgtctccgatccttgtgacgggcgtactcagggtacaacggggatgtttgattcgctaccataccgaaatgacgcatcgatggtaatgcgccgccttattcgctctctgcccgacgcgaaagcagttattggtgtggcgagttgcgataaggggcttccggccaccatgatggcactcgccgcgcagcacaacatcgcaaccgtgctggtccccggcggcgcgacgctgcccgcaaaggatggagaagacaacggcaaggtgcaaaccattggcgcacgcttcgccaatggcgaattatctctacaggacgcacgccgtgcgggctgtaaagcctgtgcctcttccggcggcggctgtcaatttttgggcactgccgggacatctcaggtggtggccgaaggattgggactggcaatcccacattcagccctggccccttccggtgagcctgtgtggcgggagatcgccagagcttccgcgcgagctgcgctgaacctgagtcaaaaaggcatcaccacccgggaaattctcaccgataaagcgatagagaatgcgatgacggtccatgccgcgttcggtggttcaacaaacctgctgttacacatcccggcaattgctcaccaggcaggttgccatatcccgaccgttgatgactggatccgcatcaacaagcgcgtgccccgactggtgagcgtactgcctaatggcccggtttatcatccaacggtcaatgcctttatggcaggtggtgtgccggaagtcatgttgcatctgcgcagcctcggattgttgcatgaagacgttatgacggttaccggcagcacgctgaaagaaaacctcgactggtgggagcactccgaacggcgtcagcggttcaagcaactcctgctcgatcaggaacaaatcaacgctgacgaagtgatcatgtctccgcagcaagcaaaagcgcgcggattaacctcaactatcaccttcccggtgggcaatattgcgccagaaggttcggtgatcaaatccaccgccattgacccctcgatgattgatgagcaaggtatctattaccataaaggtgtggcgaaggtttatctgtccgagaaaagtgcgatttacgatatcaaacatgacaagatcaaggcgggcgatattctggtcattattggcgttggaccttcaggtacagggatggaagaaacctaccaggttaccagtgccctgaagcatctgtcatacggtaagcatgtttcgttaatcaccgatgcacgtttctcgggcgtttctactggcgcgtgcatcggccatgtggggccagaagcgctggccggaggccccatcggtaaattacgcaccggggatttaattgaaattaaaattgattgtcgcgagcttcacggcgaagtcaatttcctcggaacccgtagcgatgaacaattaccttcacaggaggaggcaactgcaatattaaatgccagacccagccatcaggatttacttcccgatcctgaattgccagatgatacccggctatgggcaatgcttcaggccgtgagtggtgggacatggaccggttgtatttatgatgtaaacaaaattggcgcggctttgcgcgattttatgaataaaaactga

SEQ ID NO: 14 - DNA, 1149 bp, Escherichia coli
  source 1..1149: mol_type = other DNA
atggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaa

SEQ ID NO: 15 - DNA, 1158 bp, Zymomonas mobilis
  source 1..1158: mol_type = other DNA
atgctcaattttgattattataatccgacccatattgtttttggtaaaggccgtattgcacagcttgatacgcttttgtcgaaagatgcccgtgttttagtcctttatggcggttctagcgctcaaaagaccggcactttggatgaagtcagaaaagcccttggtgaccgcacttattttgaatttggtggtatcgaacccaatcccagctatgaaactctgatgaaagccgtcgagcaggtcaaacaggaaaaggttgattttctgttggctgttgggggtggatcagtcattgatggcaccaaattcgtagcagccgccgtgccttatgaaggtgagccttgggaaattctggaaacggatggcaagaaaatcaaggaagccttgcccgttggaaccgttttaaccctgccggctaccggctccgaaatgaaccggaatagtgtggtaacccgcaagtcaatcaaatcgaaacggggtttccataatgaccatgtttttccggttttctcgattcttgacccgacaaaggtttacactttgccgccgcgccagctggcaaatggggttgtcgattcctttattcatatcaccgaacaatatctgacctatccggttgatggtatggttcaggatgaatttgcagaaggtctgctccgcaccttaatcaaaattggccctgaattgctgaaagatcagaagaattatgatctagccgccaattttatgtggacggcgaccttggctttgaatggcttgatcggtgccggtgtgccacaggattgggcaacccatatggtcggtcatgaattgacggccgcctttggtatcgatcatggtcggacattggcgattattctcccttcgctgttgcaaaatcagagagaggccaagaaaggcaagctgctgcaatatgccaagaatgtttggcatattgatcagggctctgatgatgaacggattgatgccgcaatcgaaaagactcgtcatttctttgaatcattaggtattccgacccatctgaaagattatgatgttggggaagagtctattgatatgctggtcaaggaattagaagcgcatggcatgtcccagctaggcgaacataaggctattacgccagaggttagccgcgctattctgcttgcaagcttgtaa

SEQ ID NO: 16 - DNA, 201 bp, unidentified
  source 1..201: mol_type = other DNA
tgtctatactccagttactcaatacgtaacaataatcagtttatcctaactatagaatcgcatgagaagcgataacgtttcaccataagcaatatattcattgcaacagtggaattgccttatgcgtcaaggaaggatagatcattgacggactgagttcaaaaagagactcgtctaaaagattttaagaaaggtttcgat

SEQ ID NO: 17 - DNA, 50 bp, synthetic construct
  source 1..50: mol_type = other DNA
tgtctatactccagttactcaatacgtaacaataatcagtttatcctaac

SEQ ID NO: 18 - DNA, 32 bp, synthetic construct
  source 1..32: mol_type = other DNA
atcgaaacctttcttaaaatcttttagacgag

SEQ ID NO: 19 - DNA, 47 bp, synthetic construct
  source 1..47: mol_type = other DNA
attttaagaaaggtttcgatatgaccattgagaaaattttcaccccg

SEQ ID NO: 20 - DNA, 46 bp, synthetic construct
  source 1..46: mol_type = other DNA
ggcattttctcctctttaatttaaattccgagcgcttttttaccgg

SEQ ID NO: 21 - DNA, 38 bp, synthetic construct
  source 1..38: mol_type = other DNA
tttaaattaaagaggagaaaatgccgcagtccgcgttg

SEQ ID NO: 22 - DNA, 43 bp, synthetic construct
  source 1..43: mol_type = other DNA
ttcattttctcctctttaattcagcaaagcttgagctgttgca

SEQ ID NO: 23 - DNA, 46 bp, synthetic construct
  source 1..46: mol_type = other DNA
gctgaattaaagaggagaaaatgaacaactttaatctgcacacccc

SEQ ID NO: 24 - DNA, 40 bp, synthetic construct
  source 1..40: mol_type = other DNA
cagcggccgctactagtttagcgggcggcttcgtatatac

SEQ ID NO: 25 - DNA, 38 bp, synthetic construct
  source 1..38: mol_type = other DNA
attttaagaaaggtttcgatatgagcaatcgcaccccg

SEQ ID NO: 26 - DNA, 48 bp, synthetic construct
  source 1..48: mol_type = other DNA
ggcattttctcctctttaatttaatgattatgacgcggcaatttagcc

SEQ ID NO: 27 - DNA, 48 bp, synthetic construct
  source 1..48: mol_type = other DNA
attttaagaaaggtttcgatatgtctgttcgcaatatttttgctgacg

SEQ ID NO: 28 - DNA, 51 bp, synthetic construct
  source 1..51: mol_type = other DNA
ggcattttctcctctttaattcagtttttattcataaaatcgcgcaaagcc

SEQ ID NO: 29 - DNA, 50 bp, synthetic construct
  source 1..50: mol_type = other DNA
gctgaattaaagaggagaaaatggctaacagaatgattctgaacgaaacg

SEQ ID NO: 30 - DNA, 43 bp, synthetic construct
  source 1..43: mol_type = other DNA
cagcggccgctactagtttaccaggcggtatggtaaagctcta

SEQ ID NO: 31 - DNA, 57 bp, synthetic construct
  source 1..57: mol_type = other DNA
gctgaattaaagaggagaaaatgctcaattttgattattataatccgacccatattg

SEQ ID NO: 32 - DNA, 42 bp, synthetic construct
  source 1..42: mol_type = other DNA
cagcggccgctactagtttacaagcttgcaagcagaatagcg

(19) United States
(12) Patent Application Publication
(10) Pub. No.: US 2024/0301448 A1
(43) Pub. Date: Sep. 12, 2024

(54) RECOMBINANT ZYMOMONAS MOBILIS FOR PRODUCING ETHYLENE GLYCOL, METHOD AND USES THEREOF

(71) Applicant: Hubei University, Wuhan (CN)

(72) Inventors: Shihui YANG, Wuhan (CN); Xiongying YAN, Wuhan (CN); Mian LI, Wuhan (CN); Xia WANG, Wuhan (CN); Qiaoning HE, Wuhan (CN)

(73) Assignee: Hubei University, Wuhan (CN)

(21) Appl. No.: 18/401,511

(22) Filed: Dec. 31, 2023

(30) Foreign Application Priority Data
Mar. 10, 2023 (CN) 202310246847.7

(51) Int. Cl. C12P 7/18; C12N 9/02; C12N 9/04; C12N 9/88; C12N 15/74

(52) U.S. Cl. CPC C12P 7/18; C12N 9/0006; C12N 9/0008; C12N 9/88; C12N 15/74; C12Y 101/01001; C12Y 101/01175; C12Y 102/01021; C12Y 401/02; C12Y 402/01082

(57) ABSTRACT

Recombinant Zymomonas mobilis for producing ethylene glycol, method and uses thereof are provided. The recombinant Zymomonas mobilis carries and expresses genes related to a synthesis pathway of xylonic acid and genes related to a synthesis pathway of ethylene glycol.

### CROSS-REFERENCE TO RELATED APPLICATIONS

This disclosure claims priority to Chinese Patent Application NO: 202310246847.7, filed with China Intellectual Property Office on Mar. 10, 2023, the entire contents of which are incorporated herein by reference.

### SEQUENCE LISTING

The sequence listing xml file submitted herewith, named “WI_US23_12323_P_Sequence_Listing.xml”, created on Apr. 18, 2024, and having a file size of 41,021 bytes, is incorporated by reference herein.

### TECHNICAL FIELD

This disclosure relates to Zymomonas mobilis. Specifically, this disclosure relates to recombinant Zymomonas mobilis for producing ethylene glycol, method and uses thereof.

### BACKGROUND

The statements herein provide background information relevant to the present disclosure only and do not necessarily constitute prior art.

Ethylene glycol, the simplest diol, is an economically important platform chemical that is steadily increasing in global demand and is widely used as a monomer for esterification and etherification of unsaturated polyester resins, polyethers and polyurethanes. A traditional two-step process is used for producing ethylene glycol. The process includes fermenting glucose to obtain ethanol, and chemically converting the ethanol to ethylene glycol. This process is inefficient, energy-consuming and environmentally unfriendly. Therefore, biosynthesis is a highly selective, low energy demand and environmentally friendly method for producing ethylene glycol. For example, metabolically engineered microorganisms, such as Escherichia coli, Saccharomyces cerevisiae, Corynebacterium glutamicum etc., are able to use xylose for producing ethylene glycol. However, the production process of these microorganisms are strictly aerobic, the cost of the production is high, and a large number of by-products would be produced.

Zymomonas mobilis, a natural ethanol-producing and facultative anaerobic Gram-negative bacterium, has unique Entner-Doudoroff (ED) metabolic pathway and high sugar fermentation efficiency. Moreover, as an ideal industrial cell factory, Zymomonas mobilis has the characteristics such as, high ethanol production, low biomass production, strong ethanol tolerance, high osmotic pressure resistance, and no need for additional oxygen during the fermentation process. At present, the production of PHB, 2,3 butanediol, isobutanol and lactic acid have been realized in Zymomonas mobilis. Moreover, benefit by its high tolerance to lignocellulose hydrolysate, the production of ethanol from cellulose has already been commercialized in Zymomonas mobilis. Meanwhile, the mechanisms related to the tolerance of inhibitors in the lignocellulosic hydrolysates of Zymomonas mobilis has also been maturely studied. Additionally, by means of synthetic biology and metabolic engineering, Zymomonas mobilis could be modified into chassis cells that produce different platform compounds from the lignocellulosic hydrolysates.

### SUMMARY

Embodiments disclose a recombinant strain from Zymomonas mobilis for producing ethylene glycol. The recombinant strain carries and expresses genes related to a synthesis pathway of xylonic acid and genes related to a synthesis pathway of ethylene glycol.

Embodiments disclose a recombinant plasmid for expressing ethylene glycol. The recombinant plasmid carries sequences of a gene xdh, a gene yagF, a gene yagE and a gene yqhD which are successively connected. Herein, the gene xdh is expressed with a promoter Ppdc. The gene yagF, the gene yagE and the gene yqhD are connected by a sequence of RBS and are expressed with a promoter Peno.

Embodiments disclose a recombinant plasmid for expressing ethylene glycol. The recombinant plasmid carries sequences of a gene xdh, a gene xyID, a gene yagE and a gene yqhD which are successively connected. Herein, the gene xdh is expressed with a promoter Ppdc. The gene xyID, the gene yagE and the gene yqhD are connected by using a sequence of RBS and are expressed with a promoter Peno.

Embodiments disclose a recombinant plasmid for expressing ethylene glycol. The recombinant plasmid carries sequences of a gene xdh, a gene yjhG, a gene yagE and a gene yqhD which are successively connected. Herein, the gene xdh is expressed by using a promoter Ppdc. The gene yjhG, the gene yagE and the gene yqhD are connected by using a sequence of RBS and are expressed with a promoter Peno.

Embodiments disclose a recombinant plasmid for expressing ethylene glycol. The recombinant plasmid carries sequences of a gene xdh, a gene yagF, a gene yagE and a gene fucO which are successively connected. Herein, the gene xdh is expressed by using a promoter Ppdc. The gene yagF, the gene yagE and the gene fucO are connected by using a sequence of RBS and are expressed with a promoter Peno.

Embodiments disclose a recombinant plasmid for expressing ethylene glycol. The recombinant plasmid carries sequences of a gene xdh, a gene yagF, a gene yagE and a gene ZMO1771 which are successively connected. Herein, the gene xdh is expressed by using a promoter Ppdc. The gene yagF, the gene yagE and the gene ZMO1771 are connected by using a sequence of RBS and are expressed with a promoter Peno.

Embodiments disclose a recombinant strain from Zymomonas mobilis for producing ethylene glycol. The recombinant strain has a transferred recombinant plasmid that carries a gene xdh, a gene yagF, a gene yagE and a gene yqhD.

Embodiments disclose a recombinant strain from Zymomonas mobilis for producing ethylene glycol. The recombinant strain has a transferred recombinant plasmid that carries a gene xdh, a gene xylD, a gene yagE and a gene yqhD.

Embodiments disclose a recombinant strain from Zymomonas mobilis for producing ethylene glycol. The recombinant strain has a transferred recombinant plasmid that carries a gene xdh, a gene yjhG, a gene yagE and a gene yqhD.

Embodiments disclose a recombinant strain from Zymomonas mobilis for producing ethylene glycol. The recombinant strain has a transferred recombinant plasmid that carries a gene xdh, a gene yagF, a gene yagE and a gene fucO.

Embodiments disclose a recombinant strain from Zymomonas mobilis for producing ethylene glycol. The recombinant strain has a transferred recombinant plasmid that carries a gene xdh, a gene yagF, a gene yagE and a gene ZMO1771.

Embodiments disclose a method of preparing recombinant strains from Zymomonas mobilis for producing ethylene glycol. The method includes overexpressing genes involved in a synthesis path way of xylonic acid, and overexpressing genes involved in a synthesis pathway of ethylene glycol in Zymomonas mobilis. The genes involved in a synthesis pathway of xylonic acid include a gene xdh coding for xylose dehydrogenase. The genes involved in a synthetic pathway of ethylene glycol include coding genes yagF for xylonate dehydratase, yagE for aldolase, yqhD for glycolaldehyde reductase, xyID and yjhG for xylonate dehydratase, fucO and ZMO1771 for alcohol dehydrogenase.

Embodiments disclose a use of a recombinant strain from Zymomonas mobilis as embodiments described above for producing ethylene glycol.

Embodiments disclose a use of a recombinant strain prepared by the method as embodiments described above for producing ethylene glycol.

### BRIEF DESCRIPTION OF DRAWINGS

FIG. 1 depicts a schematic diagram of preparing a recombinant plasmid pXA1 according to embodiments.

FIG. 2A shows test results of glucose consumption and ethanol production without CaCO₃ by an intermediate strain (Z. mobilis XA1) according to embodiments.

FIG. 2B shows test results of glucose consumption and ethanol production with CaCO₃ by an intermediate strain (Z. mobilis XA1) according to embodiments.

FIG. 2C shows test results of producing xylonic acid by an intermediate strain (Z. mobilis XA1) according to embodiments.

FIG. 2D shows test results of pH variation with or without CaCO₃ of an intermediate strain (Z. mobilis XA1) according to embodiments.

FIG. 3 depicts a Dahms pathway of preparing a recombinant strain EG2 from Zymomonas mobilis provided according to embodiments.

FIG. 4 depicts a schematic diagram of preparing five recombinant plasmids for ethylene glycol synthesis provided according to embodiments.

FIG. 5 shows the test results of fermenting property of five recombinant strains for ethylene glycol synthesis provided according to embodiments.

FIG. 6A shows test results of glucose consumption and ethanol production of a recombinant strain Zymomonas mobilis EG2 in xylose mother liquor according to embodiments.

FIG. 6B shows test results of productions of ethylene glycol and xylonic acid of a recombinant strain Zymomonas mobilis EG2 in xylose mother liquor according to embodiments.

### DETAILED DESCRIPTION OF THE EMBODIMENTS

Embodiments provides recombinant strains from Zymomonas mobilis for producing ethylene glycol. These recombinant strains are genetic Zymomonas mobilis ZM4s that carry and express genes related to synthesis pathways of xylonic acid and/or ethylene glycol. To solve the problem of oxygen demand of ethylene glycol's production, a Dahms Pathway, which takes xylonic acid as a main intermediate, is transferred into an original strain Zymomonas mobilis ZM4 to obtain recombinant strains in this disclosure. These recombinant strains are capable of producing ethylene glycol by utilizing xylose. And these recombinant strains are able to produce ethylene glycol with high yield in pure sugar mother liquor or toxic xylose mother liquor, by comparing xylonate dehydratase with glycolaldehyde reductase from different sources. The yield of ethylene glycol of these recombinant strains are able to reach 3.33 g/L fermentation broth.

These recombinant strains use their own powerful restriction-modification systems to have more antiviral capabilities than commonly engineered strains such as E. coli.

Since Z. mobilis is a facultative anaerobic microorganism, the fermentation of these recombinant strains based on Z. mobilis requires no dissolved oxygen control equipment, which can effectively reduce the production cost.

Since Z. mobilis is a growing and metabolically uncoupled microorganism, these recombinant strains based on Z. mobilis can efficiently convert the carbon source to the final metabolite ethanol, and are also suitable for producing other compounds with potentially high conversion rates.

In some embodiments, the xylonic acid synthesis pathway-related genes include a xylose dehydrogenase xdh gene.

In some embodiments, the ethylene glycol synthesis pathway-related genes include a coding gene yagF for xylonate dehydratase, a coding gene yagE for aldolase, a coding gene yqhD for glycolaldehyde reductase, coding genes xyID and yjhG for xylonate dehydratase, coding genes fucO and ZMO1771 for alcohol dehydrogenase.

Embodiments disclose a recombinant strain from Zymomonas mobilis for producing ethylene glycol. The recombinant strain has a transferred recombinant plasmid that carries a gene xdh, a gene yagF, a gene yagE and a gene yqhD.

Embodiments disclose a recombinant strain from Zymomonas mobilis for producing ethylene glycol. The recombinant strain has a transferred recombinant plasmid that carries a gene xdh, a gene xylD, a gene yagE and a gene yqhD.

Embodiments disclose a recombinant strain from Zymomonas mobilis for producing ethylene glycol. The recombinant strain has a transferred recombinant plasmid that carries a gene xdh, a gene yjhG, a gene yagE and a gene yqhD.

Embodiments disclose a recombinant strain from Zymomonas mobilis for producing ethylene glycol. The recombinant strain has a transferred recombinant plasmid that carries a gene xdh, a gene yagF, a gene yagE and a gene fucO.

Embodiments disclose a recombinant strain from Zymomonas mobilis for producing ethylene glycol. The recombinant strain has a transferred recombinant plasmid that carries a gene xdh, a gene yagF, a gene yagE and a gene ZMO1771.

This Disclosure Also Provides Recombinant Plasmids for Expressing Ethylene Glycol.

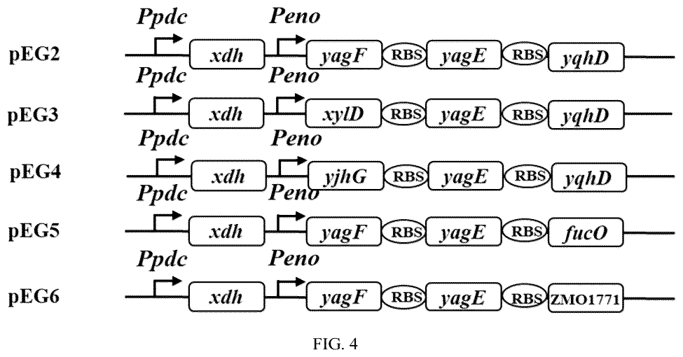

FIG. 4 shows a recombinant plasmid (pEG2) that carries a sequence of a gene xdh, a gene yagF, a gene yagE and a gene yqhD successively connected. Herein, the xdh is expressed with a promoter Ppdc. The gene yagF, the gene yagE and the gene yqhD are connected by an RBS and are expressed with a promoter Peno.

FIG. 4 shows a recombinant plasmid (pEG3) that carries a sequence of a gene xdh, a gene xylD, a gene yagE, and a gene yqhD successively connected Herein, the gene xdh is expressed with a promoter Ppdc. The gene xyID, the gene yagE, and the gene yqhD are connected with a sequence of RBS and are expressed with a promoter Peno.

FIG. 4 also shows a recombinant plasmid (pEG4) that carries a sequence of a gene xdh, a gene yjhD, a gene yagE and a gene yqhD successively connected. Herein, the gene xdh is expressed with a promoter Ppdc. The gene yjhD, the gene yagE and the gene yqhD are connected with a sequence of RBS and are expressed with a promoter Peno.

FIG. 4 also shows a recombinant plasmid (pEG5) that carries a sequence of a gene xdh, a gene yagF, a gene yagE and a gene fucO successively connected. Herein, the gene xdh is expressed with a promoter Ppdc. The gene yagF, the gene yagE and the gene fucO are connected with a sequence of RBS and are expressed with a promoter Peno.

FIG. 4 also shows a recombinant plasmid (pEG6) carries a sequence of a gene xdh, a gene yagF, a gene yagE and a gene ZMO1771 successively connected. Herein, the gene xdh is expressed with a promoter Ppdc. The gene yagF, the gene yagE and the gene ZMO1771 are connected with a sequence of RBS and are expressed with a promoter Peno.

Embodiments also provide a method of preparing recombinant strains from Zymomonas mobilis for producing ethylene glycol. The method includes: overexpressing genes involved in a synthetic pathway of xylonic acid in Zymomonas mobilis, and overexpressing genes involved in a synthetic pathway of ethylene glycol in Zymomonas mobilis. The genes involved in a synthetic pathway of xylonic acid include a gene xdh coding for xylose dehydrogenase. The genes involved in a synthesis pathway of ethylene glycol include coding genes yagF for xylonate dehydratase, yagE for aldolase, yqhD for glycolaldehyde reductase, xyID and yjhG for xylonate dehydratase, fucO and ZMO1771 for alcohol dehydrogenase.

In some embodiments, recombinant plasmids have been constructed to overexpress genes involved in a synthetic pathway of xylonic acid, and genes involved in a synthesis pathway of ethylene glycol.

Some embodiments provide a method of preparing a recombinant strain from Zymomonas mobilis for producing ethylene glycol. The method includes: obtaining a promoter Ppdc, a gene xdh, a gene yagF, a gene yagE, a gene yqhD and a promoter Peno; connecting and inserting the promoter Ppdc and the gene xdh into a basal plasmid pEZ15A to obtain a plasmid for overexpressing xylose dehydrogenase; transferring the plasmid for overexpressing xylose dehydrogenase into Zymomonas mobilis ZM4 to obtain an intermediate strain; sequentially connecting the promoter Peno, the gene yagF, the gene yagE and the gene yqhD to obtain a fusion sequence, herein the gene yagF, the gene yagE and the gene yqhD are connected by a sequence of RBS; inserting the fusion sequence into the plasmid for overexpressing xylose dehydrogenase to obtain a recombinant plasmid; transferring the recombinant plasmid into the intermediate strain to obtain the recombinant strain.

Some embodiments provide a method of preparing a recombinant strain from Zymomonas mobilis for producing ethylene glycol. The method includes: obtaining a promoter Ppdc, a gene xdh, a gene xylD, a gene yagE, a gene yqhD and a promoter Peno; connecting and inserting the promoter Ppde and the gene xdh into a basal plasmid pEZ15A to obtain a plasmid for overexpressing xylose dehydrogenase; transferring the plasmid for overexpressing xylose dehydrogenase into Zymomonas mobilis ZM4 to obtain an intermediate strain; sequentially connecting the promoter Peno, the gene xyID, the gene yagE and the gene yqhD to obtain a fusion sequence, herein the gene xyID, the gene yagE and the gene yqhD are connected by a sequence of RBS; inserting the fusion sequence into the plasmid for overexpressing xylose dehydrogenase to obtain a recombinant plasmid; transferring the recombinant plasmid into the intermediate strain to obtain the recombinant strain.

Some embodiments provide a method of preparing a recombinant strain from Zymomonas mobilis for producing ethylene glycol. The method includes: obtaining a promoter Ppdc, a gene xdh, a gene yjhG, a gene yagE, a gene yqhD and a promoter Peno; connecting and inserting the promoter Ppdc and the gene xdh into a basal plasmid pEZ15A to obtain a plasmid for overexpressing xylose dehydrogenase; transferring the plasmid for overexpressing xylose dehydrogenase into Zymomonas mobilis ZM4 to obtain an intermediate strain; sequentially connecting the promoter Peno, the gene yjhG, the gene yagE and the gene yqhD to obtain a fusion sequence, herein the gene yjhG, the gene yagE and the gene yqhD are connected by a sequence of RBS; inserting the fusion sequence into the plasmid for overexpressing xylose dehydrogenase to obtain a recombinant plasmid; transferring the recombinant plasmid into the intermediate strain to obtain the recombinant strain.

Some embodiments provide a method of preparing a recombinant strain from Zymomonas mobilis to producing ethylene glycol. The method includes: obtaining a promoter Ppdc, a gene xdh, a gene yagF, a gene yagE, a gene fucO and a promoter Peno; connecting and inserting the promoter Ppdc and the gene xdh into a basal plasmid pEZ15A to obtain a plasmid for overexpressing xylose dehydrogenase; transferring the plasmid for overexpressing xylose dehydrogenase into Zymomonas mobilis ZM4 to obtain an intermediate strain; sequentially connecting the promoter Peno, the gene yagF, the gene yagE and the gene fucO to obtain a fusion sequence, herein the gene yagF, the gene yagE and the gene fucO are connected by a sequence of RBS; inserting the fusion sequence into the plasmid for overexpressing xylose dehydrogenase to obtain a recombinant plasmid; transferring the recombinant plasmid into the intermediate strain to obtain the recombinant strain.

Some embodiments provide a method of preparing a recombinant strain from Zymomonas mobilis for producing ethylene glycol. The method includes: obtaining a promoter Ppdc, a gene xdh, a gene yagF, a gene yagE, a gene ZMO1771 and a promoter Peno; connecting and inserting the promoter Ppdc and the gene xdh into a basal plasmid pEZ15A to obtain a plasmid for overexpressing xylose dehydrogenase; transferring the plasmid for overexpressing xylose dehydrogenase into Zymomonas mobilis ZM4 to obtain an intermediate strain; sequentially connecting the promoter Peno, the gene yagF, the gene yagE and the gene ZMO1771 to obtain a fusion sequence, herein the gene yagF, the gene yagE and the gene ZMO1771 are connected by a sequence of RBS; inserting the fusion sequence into the plasmid for overexpressing xylose dehydrogenase to obtain a recombinant plasmid; transferring the recombinant plasmid into the intermediate strain to obtain the recombinant strain.

Embodiments of this disclosure also provide uses of these recombinant plasmids of Z. mobilis described above for producing ethylene glycol.

Embodiments of this disclosure also provide uses of these methods of preparing these recombinant plasmids of Z. mobilis described above for producing ethylene glycol.

The present disclosure will be illustrated below with more specific examples, but is not construed as limiting the embodiments of this disclosure.

### Example 1: Prepare an Intermediate Strain (Z. mobilis XA1)

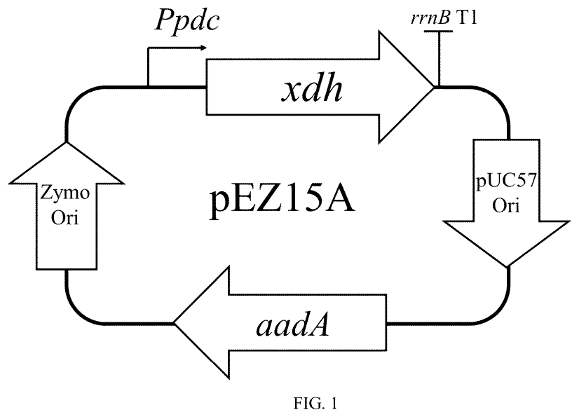

One important step of the synthesis from xylose to ethylene glycol is the synthesis of xylonic acid. And the synthesis from xylose to xylonic acid is achieved through expressing of a coding gene of xylose dehydrogenase derived from Paraburkholderia xenovorans and an enzymatic reaction. Z. mobilis XA1 can be prepared by transferring a plasmid pXA1 shown in FIG. 1 into Z. mobilis, and controlling the expression of xylose dehydrogenase with a strong promoter.

(1) Prepare the Plasmid pXA1

A sequence of gene xdh (shown in SEQ ID NO. 1, derived from Paraburkholderia xenovorans) and a sequence of strong promoter Ppdc (shown in SEQ ID NO. 2, derived from the genome of Zymomonas mobilis ZM4) were amplified by PCRs respectively. A basal plasmid pEZ15A (Yang, S. et al. (2016). Metabolic engineering of Zymomonas mobilis for 2,3-butanediol production from lignocellulosic biomass sugars. Biotechnology for biofuels, 9(1), 189.) was amplified in reverse by a PCR.

Herein, the PCR procedure was set as: 98° C. pre-denaturation for 2 min; 98° C. denaturation for 10s, 55° C. annealing for 10s, 72° C. extension (set according to fragment length at 10s/kb) for a total of 30 cycles; 72° C. for 5 min after the end of the cycle reaction. The PCR product was stored at −20° C. after purification. And the other conditions of PCR were set as follows:

TABLE 1
PCR reaction system for sequence of gene xdh

| Component | Dosage(μL) |
|---|---|
| F-primer (10 μM) | 0.5 |
| R-primer (10 μM) | 0.5 |
| PRIMESTAR ® DNA Polymerase (Takara) | 10 |
| Template (5~10 ng) | X |
| ddH₂O | To 20 |
| Total volume | 20 |

### Primers:

Ppdc-F:
SEQ ID NO. 3
attcgcggccgcttctagagtatcgctcatgatcgcggcat,
Ppdc-R:
SEQ ID NO. 4
ggatagctcaacagatacattgcttactccatatattcaaaacactatgt
ctgaatc,
Xdh-F:
SEQ ID NO. 5
atgtatctgttgagctatccggaacag,
Xdh-R:
SEQ ID NO. 6
ttattcaccataccaaccagcatcaacaaaa,

A fragment of xdh was mixed with a fragment of the basal plasmid pEZ15A in a ratio of 3:1 and configured to a reaction system as shown in Table 2. After 5 minutes of ice bath, the production of reaction was transferred to competent cells of E. coli by a general process.

Positive colonies were picked on plates with containing Spectinomycin. Subsequently, these positive colonies were verified by colony PCRs with primers 15A-fwd (gtaaaacgacggccagt, SEQ ID NO. 7)/15A-rev (gtcatagctgtttcctg, SEQ ID NO. 8). A procedure of the colony PCR was set as: 98° C. pre-denaturation for 3 min; 98° C. denaturation for 10s, 55° C. annealing for 10s, 72° C. extension for 80s for 30 cycles. The bands of the colony that was consistent with the expectations, were then verified by sequencing. Finally, the plasmid pXA1 could be extracted from the verified positive clones.

TABLE 2
Ligation reaction system of xdh with pEZ15A

| Component | Dosage | |
|---|---|---|
| fragment of xdh | 0.12 | pM |
| pEZ15A | 0.04 | pM |
| 10 × Buffer 4 (Thermo) | 0.5 | μL |
| T5 Exonuclease | 0.5 | U |

| | |
|---|---|
| ddH₂O | To 5 μL |

### (2) Prepare the Intermediate Strain

The process of preparing the intermediate strain included preparing competent cells of Z. mobilis ZM4 (Z. mobilis subsp. mobilis ZM4 ATCC3182, ATCC). 100 μL frozen bacteria of Z. mobilis ZM4 was removed from a −80° C. refrigerator firstly, and inoculated into 1 mL RMG5 loaded in a freezing tube and culturing at 30° C. to activate the strain. After the culture became cloudy, the cloudy culture was shifted into 200 mL liquid medium RMG5 loaded in a 250 ml bottle with blue cover, and the initial OD 600 nm was set within 0.025˜0.3 in a 30° C. incubator. When OD600 nm of the culture exceeded 0.3, the bacteria was collected by centrifugation of 100 rpm at room temperature. The bacteria was washed once with sterile water and twice with 10% glycerin, and was slowly resuspended with 1-2 mL 10% glycerin, and finally 55 μL bacteria was packed into one 1.5 mL EP tube.

The process of preparing the intermediate strain also included the transfer of the plasmid pXA1 into the competent cells. Firstly, 1 mg of the plasmid pXA1 was added and gently mixed into 55 μL competent cells loaded in a 1.5 mL EP tube, and shifted into a 1 mm electroporation cuvette. And then the electroporation cuvette was placed into an electroporation instrument to electro-transfer. Herein, the electro-transformation conditions were set as: 200 Ω, capacitor: 25 μF, voltage: 1.6 KV. And 1 mL RMG5 of liquid medium was added into the electroporation cuvette after electro-transferring, mixed well and then shifted to a sterile EP tube, sealed and incubated in a 30° C. thermostatic incubator for 4˜6 h to get the transferred strains. And 100 μL of the transferred strains solution was evenously coated on a plate (that is plate containing 100 μg/mL Spectinomycin in RMG5, named RMG5+Spe); sealed and anastrophic incubated in a 30° C. thermostatic incubator.

Positive colonies were picked and verified by colony PCR with primers 15A-fwd and 15A-rev. The reaction system and procedure of the colony PCR were set as same as the above steps. The positive clones were activated in the medium of RMG5+Spe, and preserved by glycerin.

### (3) Test of Yield of Xylonic Acid for the Intermediate Strain

The intermediate strain XA1 was activated in medium RMG5, and cultivated to get the seeds.

The seeds were inoculated into a fermentation medium (named rich medium, RM, 10 g/L yeast extract, 20 g/L Glucose, 20 g/L Xylose, 2 g/L KH₂PO₄), and fermented at 30° C., 100 rpm, and added antibiotic(spectinomycin) with a final concentration of 100 μg/mL if necessary.

The fermentation broth was centrifuged, and the supernatant was filtered by a 0.2 μm filter into an HPLC vial. The yield of xylonic acid for the intermediate strain XA1 was tested by a HPLC system (Shimadzu, Japan) consisting of BIO-RAD AMINEX® HPX-87H column (Bio-Rad, Hercules, CA, USA), a refractive index detector (RID) and a UV-detector. Herein, the temperature of column was set at 60° C., and 5 mM H₂SO₄ of solution was used as the mobile phase at a flow rate of 0.5 mL/min.

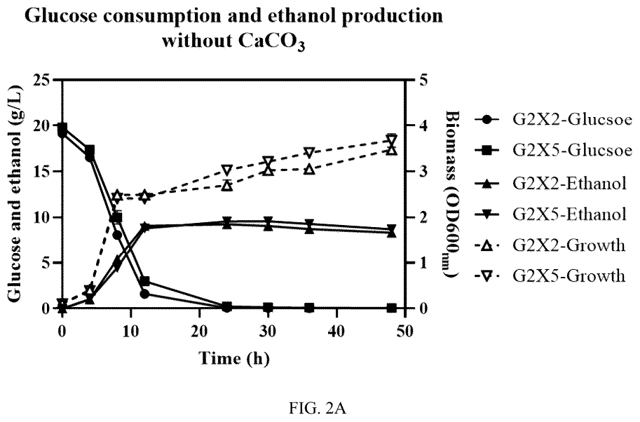

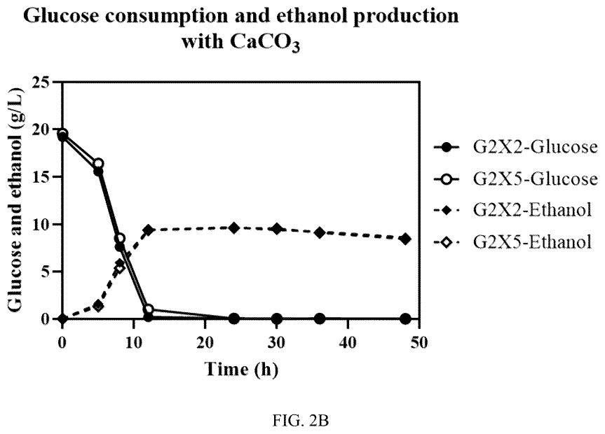

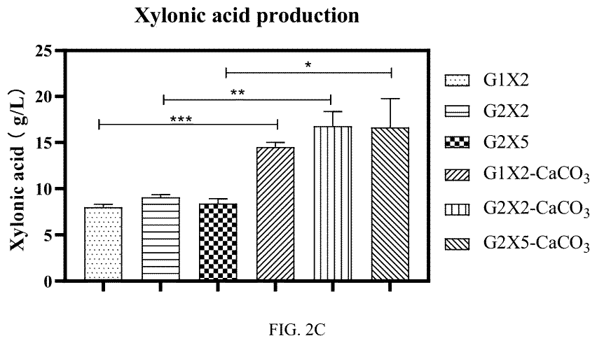

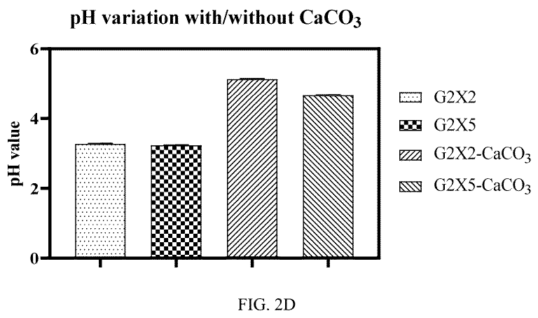

A fermentation of complete glucose consumption for 48 h in RMG2X2 medium could generate 9.67±0.01 g/L of ethanol and 9.08±0.275 g/L of xylonic acid as shown in FIG. 2A. The production of xylonic acid during the fermentation of the strain XA1 would be inhibited at the mercy of a low pH (for example 3.0) as shown in FIG. 2D. For reducing the effect of pH on the fermentation performance of the strain, CaCO₃ would be added to neutralize the produced xylonic acid. By adding CaCO₃, the strain XA1 could completely consume glucose in the medium after 12 h of fermentation, and the ethanol yield reached the maximum, and the yield of xylonic acid was significantly different at 16.78±1.58 g/L, which was much higher than the condition of no adding CaCO₃ (FIG. 2B, FIG. 2C).

### Example 2: Prepare a Recombination Strain for Producing Ethylene Glycol

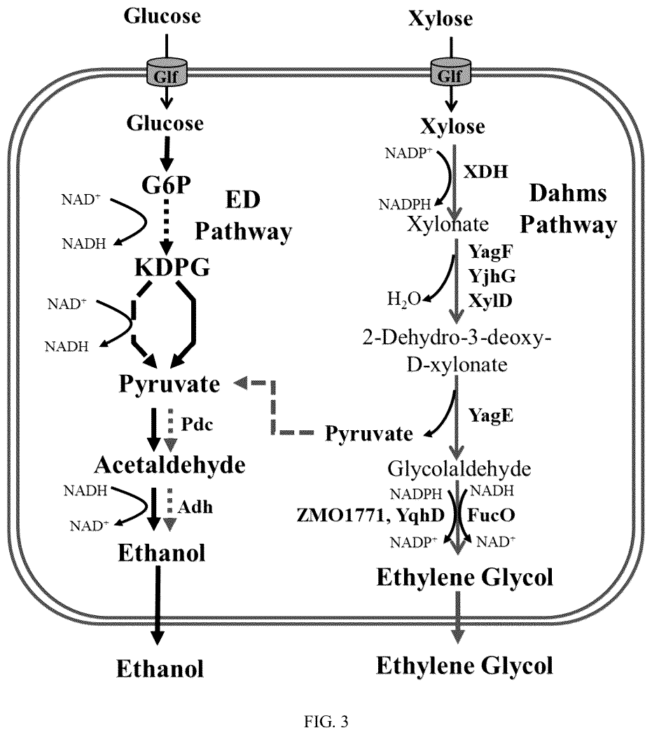

To further convert xylonic acid to ethylene glycol, an artificially designed Dahms pathway was introduced into the intermediate strain (Z. mobilis XA1) as shown in FIG. 3. In this process, a gene yagF and a gene yjhG for coding xylonate dehydratase derived from E. coli, and a gene yagE derived from E. coli for coding aldolase, and a gene yqhD and a gene fuco derived from different E. coli for coding aldehyde dehydrogenase, and a gene ZMO1771 from Z. mobilis for coding aldehyde dehydrogenase ZMO1771 were identified and tested.

### (1) Prepare Recombinant Plasmids

yagF (SEQ ID NO. 9), yagE (SEQ ID NO. 10), yqhD (SEQ ID NO. 11), xyID (SEQ ID NO. 12), yjhG (SEQ ID NO. 13), fucO (SEQ ID NO. 14), ZMO1771 (SEQ ID NO. 15) and a promoter Peno (SEQ ID NO. 16) were amplified by PCRs. Three genes (shown in FIG. 4, pEG2: yagF, yagE and yqhD; pEG3: xyID, yagE and yqhD; pEG4: yjhG, yagE and yqhD; pEG5: yagF, yagE and fucO; pEG6: yagF, yagE and ZMO1771) were respectively connected by PCRs with a sequence of RBS to get five connectors. Herein, the expressions of these three genes in the five connectors were respectively controlled by the promoter Peno. The five connectors were integrated respectively into the plasmid XA1 to assemble and construct five recombinant plasmids (pEG2˜pEG6) as shown in FIG. 4.

The PCR procedure was set as: 98° C. pre-denaturation for 2 min; 98° C. denaturation for 10s, 55° C. annealing for 10s, 72° C. extension (set according to fragment length at 10s/kb) for a total of 30 cycles; 72° C. for 5 min after the end of the cycle reaction. And the product of PCR was stored at −20° C. after purification. And the PCR reaction system was set as Table 3.

TABLE 3
PCR reaction system of yagF, yagE,
yqhD, xylD, yjhG, fucO, ZMO1771

| component | dosage(μL) |
|---|---|
| F-primer (10 μM) | 0.5 |
| R-primer (10 μM) | 0.5 |
| PRIMESTAR ® DNA Polymerase | 10 |
| (Takara) | |
| Template (5-10 ng) | X |
| ddH₂O | To 20 |
| Total volume | 20 |

### Primers:

Peno-F:
SEQ ID NO. 17
tgtctatactccagttactcaatacgtaacaataatcagtttatcctaa
c,
Peno-R:
SEQ ID NO. 18
atcgaaacctttcttaaaatcttttagacgag,
yagF-F:
SEQ ID NO. 19
attttaagaaaggtttcgatatgaccattgagaaaattttcaccccg,
yagF-R:
SEQ ID NO. 20
ggcattttctcctctttaatttaaattccgagcgcttttttaccgg,
yagE-F:
SEQ ID NO. 21
tttaaattaaagaggagaaaatgccgcagtccgcgttg,
yagE-R:
SEQ ID NO. 22
ttcattttctcctctttaattcagcaaagcttgagctgttgca,
yqhD-F:
SEQ ID NO. 23
gctgaattaaagaggagaaaatgaacaactttaatctgcacacccc,
yqhD-R:
SEQ ID NO. 24
cagcggccgctactagtttagcgggcggcttcgtatatac,
xylD-F:
SEQ ID NO. 25
attttaagaaaggtttcgatatgagcaatcgcaccccg,
xylD-R:
SEQ ID NO. 26
ggcattttctcctctttaatttaatgattatgacgcggcaatttagcc,
yjhG-F:
SEQ ID NO. 27
attttaagaaaggtttcgatatgtctgttcgcaatatttttgctgacg,
yjhG-R:
SEQ ID NO. 28
ggcattttctcctctttaattcagtttttattcataaaategcgcaaagc
c,
fucO-F:
SEQ ID NO. 29
gctgaattaaagaggagaaaatggctaacagaatgattctgaacgaaac
g,
fucO-R:
SEQ ID NO. 30
cagcggccgctactagtttaccaggcggtatggtaaagctcta,
ZMO1771-F:
SEQ ID NO. 31
gctgaattaaagaggagaaaatgctcaattttgattattataatccgacc
catattg,
ZMO1771-R:
SEQ ID NO. 32
cagcggccgctactagtttacaagcttgcaagcagaatagcg,

### (2) Prepare Five Recombinant Strains

The transformation of the five recombinant plasmids (pEG2˜pEG6) to get five recombinant strains (EG2˜pEG6) and the screening of the five recombinant strains were consistent with the construction of the intermediate strain.

### (3) Test for the Production of Ethylene Glycol of the Recombinant Strains

Strains of EG2˜EG6 were fermented respectively in a rich medium (named RMG2X2, with containing 10 g/L yeast extract, 20 g/L Glucose, 20 g/L Xylose and 2 g/L KH₂PO₄) at 30° C., 100 rpm. And spectinomycin would be added at the final concentration of 100 μg/mL if necessary.

The fermentation broth was centrifuged, and the supernatant was filtered by a 0.2 μm filter into an HPLC vial. The yield of ethylene glycol for the five recombinant strains (EG2˜pEG6) were tested by a HPLC system (Shimadzu, Japan) consisting of BIO-RAD AMINEX® HPX-87H column (Bio-Rad, Hercules, CA, USA), a refractive index detector (RID) and a UV-detector. Herein, the temperature of column was set at 60° C., and 5 mM H₂SO₄ of solution was used as the mobile phase at a flow rate of 0.5 mL/min.

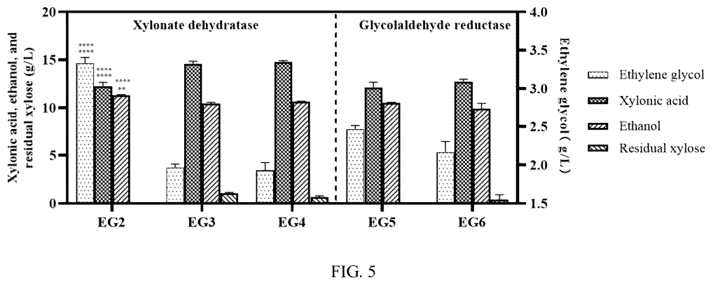

The yield of ethanol, ethylene glycol and xylonic acid of the five recombinant strains (EG2˜EG6) were tested under equal conditions. As shown in FIG. 5 and Table 4, the gene yagF coding for xylonate dehydratase and the gene yqhD coding for aldehyde dehydrogenase, could able to give the recombinant strains with best effect for producing the ethylene glycol.

TABLE 4
Test of fermenting property for EG2~pEG6

| | Consumption (g/L) | | Yield (g/L) | | |
|---|---|---|---|---|---|
| Strains | glucose | xylose | ethylene glycol | xylonic acid | ethanol |
| EG2 | 19.41 ± 0.07 | 20.36 ± 0.06 | 3.26 ± 0.07 | 17.88 ± 0.08 | 10.13 ± 0.27 |
| EG3 | 20.20 ± 0.13 | 21.13 ± 0.20 | 1.97 ± 0.04 | 12.26 ± 0.39 | 11.32 ± 0.03 |
| EG4 | 20.12 ± 1.18 | 21.23 ± 0.04 | 1.93 ± 0.10 | 14.59 ± 0.26 | 10.43 ± 0.14 |
| EG5 | 18.24 ± 0.19 | 20.39 ± 0.12 | 2.47 ± 0.05 | 14.74 ± 0.17 | 10.65 ± 0.02 |
| EG6 | 18.26 ± 0.12 | 20.84 ± 0.12 | 2.17 ± 0.14 | 12.13 ± 0.52 | 10.41 ± 0.22 |

### Example 3: Test of Recombinant Strain (EG2) in Mother Liquid Containing Xylose

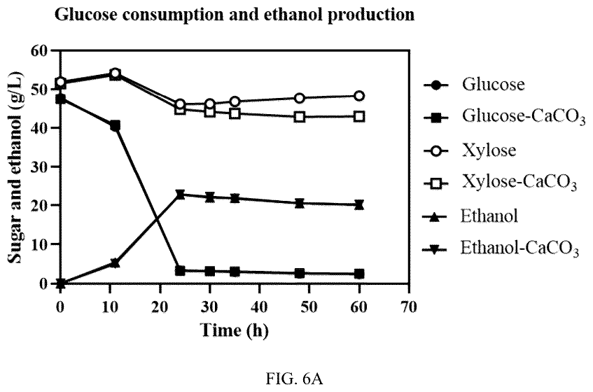

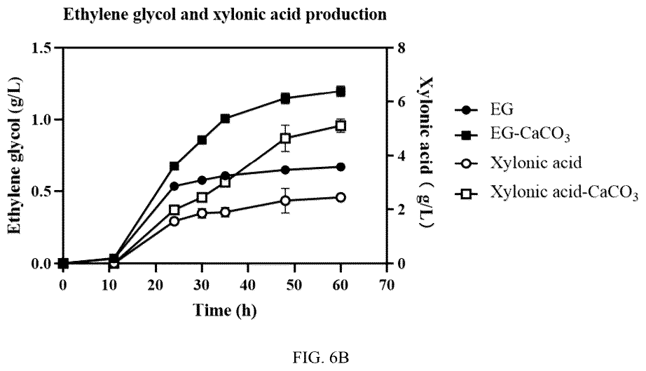

EG2 was activated and cultivated to obtain a seed solution. The seed solution was inoculated to 40 mL of fermentation medium with containing xylose mother liquor, and controlled the initial OD 600 nm of 0.1. After fermenting to the log phase at 30° C., 100 rpm, the fermentation broth was added CaCO₃ with a final concentration of 10 g/L. Samples at certain intervals of the fermentation broth were tested for the OD 600 nm and pH. And the fermentation broths at different time points were collected to detect the content of glucose, ethanol, ethylene glycol and xylonic acid with the help of HPLC as the same steps above. As shown in FIG. 6A and FIG. 6B, 1.6 g/L ethylene glycol could be produced in the recombinant strain EG2.

The above is only the preferred embodiments of this disclosure and is not intended to limit this disclosure. Any modification, equivalent replacement, improvement, etc., made within the spirit and principle of this disclosure shall be included in the scope of this disclosure.

What is claimed is:

1. A recombinant strain from Zymomonas mobilis for producing ethylene glycol, wherein the recombinant strain is transformed with a recombinant plasmid, the recombinant plasmid carries a connected sequence of a gene xdh, a gene yagF, a gene yagE; and a gene ZMO1771 wherein a nucleotide sequence of the gene xdh is the nucleotide sequence set forth in SEQ ID NO. 1, a nucleotide sequence of the gene yagF is the nucleotide sequence set forth in SEQ ID NO. 9, a nucleotide sequence of the gene yagE is the nucleotide sequence set forth in SEQ ID NO. 10, and a nucleotide sequence of the gene ZMO1771 is the nucleotide sequence set forth in SEQ ID NO. 15.

2. A method of preparing a recombinant strain from Zymomonas mobilis for producing ethylene glycol comprising:
preparing a recombinant plasmid for producing ethylene glycol, the recombinant plasmid carries a connected sequence of a gene xdh, a gene yagF, a gene yagE and a gene ZMO1771 wherein a nucleotide sequence of the gene xdh is the nucleotide sequence set forth in SEQ ID NO. 1, a nucleotide sequence of the gene yagF is the nucleotide sequence set forth in SEQ ID NO. 9, a nucleotide sequence of the gene yagE is the nucleotide sequence set forth in SEQ ID NO. 10, and a nucleotide sequence of the gene ZMO1771 is the nucleotide sequence set forth in SEQ ID NO. 15, wherein the gene xdh is expressed under a control of a promoter Ppde, wherein the gene yagF, the gene yagF, and the gene ZMO1771 are linked by a sequence of RBS and expressed under the control of a promoter Peno; and
transforming Zymomonas mobilis with the recombinant plasmid.

3. A method for using a recombinant strain prepared by the method according to claim 2 for producing ethylene glycol, wherein the method comprises steps of:
fermenting the recombinant strain prepared by the method according to claim 2 in a fermentation medium; and
filtering a fermentation broth after centrifugation to obtain the ethylene glycol.
